(19) United States
(12) Patent Application Publication
(10) Pub. No.: US 2020/0385328 A1
(43) Pub. Date: Dec. 10, 2020

(54) METHOD FOR PRODUCING BENZOYL FORMIC ACID COMPOUND AND PYRIDAZINE COMPOUND

(71) Applicant: SUMITOMO CHEMICAL COMPANY, LIMITED, Tokyo (JP)

(72) Inventors: Tadafumi MATSUNAGA, Chiba-shi (JP); Yasuhiro KATAOKA, Oita-shi (JP); Masato KAWAMURA, Oita-shi (JP); Shun TANIMURA, Takarazuka-shi (JP)

(73) Assignee: SUMITOMO CHEMICAL COMPANY, LIMITED, Tokyo (JP)

(21) Appl. No.: 16/643,327

(22) PCT Filed: Oct. 26, 2018
(86) PCT No.: PCT/JP2018/039804

§ 371 Date: Feb. 28, 2020

(30) Foreign Application Priority Data
Oct. 27, 2017 (JP) 2017-207930

(51) Int. Cl. C07C 51/275; C07D 307/60; C07D 237/14; C07D 237/12

(52) U.S. Cl. CPC C07C 51/275; C07D 307/60; C07C 59/88; C07D 237/12; C07D 237/14

(57) ABSTRACT

The present invention provides an industrially-advantageous process for preparing a benzoyl formic acid compound, and an efficient process for preparing a pyridazine compound using the same process. Specifically, the present invention provides a process for preparing a compound represented by formula (2), which comprises a step (B): a step of reacting a compound represented by formula (1) with a nitrosyl sulfuric acid in water to produce the compound represented by formula (2).

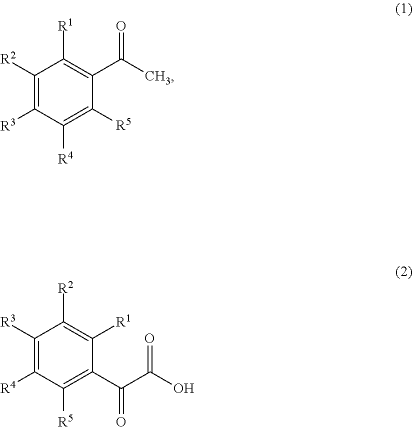

### TECHNICAL FIELD

This application claims priority to and the benefit of Japanese Patent Application No. 2017-207930 filed Oct. 27, 2017, the entire contents of which are incorporated herein by reference.

The present invention is related to a benzoyl formic acid compound, and a process for preparing a pyridazine compound using the same benzoyl formic acid as an intermediate compound, as well as a process for preparing the benzoyl formic acid compound, and a process for preparing the pyridazine compound using the same process.

### BACKGROUND ART

Patent Document 1 describes a pyridazine compound which is useful as a fungicide.

Patent s Document 2 describes that a benzoyl formic acid compound is useful as an intermediate compound for preparing the pyridazine compound.

Patent Document 3 describes that a 2′,6′-difluoroacetophenone is reacted in aqueous nitric acid solution to produce a 2′,6′-difluorobenzoyl formic acid.

### CITATION LIST

### Patent Document

Patent Document 1: WO 2005/121104

Patent Document 2: WO 2014/129612

Patent Document 3: JP 2016-169165 A

### SUMMARY OF THE INVENTION

### Problems to be Solved by Invention

The process described in the Patent Document 3 is not necessarily an industrially-sufficient process due to a lower yield of the desired product and the like.

The present invention is to provide an industrially-advantageous process for preparing a benzoyl formic acid compound, and an efficient process for preparing a pyridazine compound using the same process.

### Means to Solve Problems

The present inventors have intensively studied to solve the above-mentioned problems, and as a result, completed the present invention.

That is, the present invention includes the followings. [1] A process for preparing a below-mentioned compound represented by formula (2),

which comprises a step (B): a step of reacting a compound represented by formula (1):

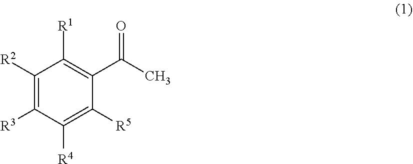

[wherein, R¹, R², R³, R⁴ and R⁵ each independently represents a fluorine atom, a chlorine atom, a bromine atom, a hydrogen atom, a hydrocarbon group, or a hydrocarbon group substituted with halogen atom(s).] (hereinafter, referred to as Compound (1))
with a nitrosyl sulfuric acid in water to produce the compound represented by formula (2):

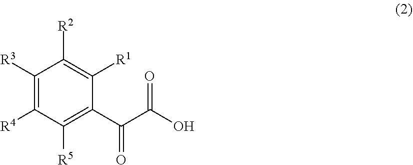

[wherein, R¹, R², R³, R⁴ and R⁵ are the same as defined above.] (hereinafter, referred to as Compound (2)).
[2] The process according to [1] wherein the step (B) is conducted in the presence of a silicon dioxide-containing inorganic material.
[3] A process for preparing a below-mentioned compound represented by formula (2):

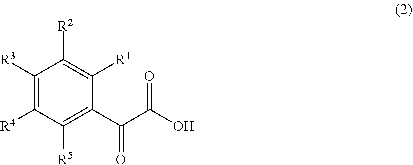

[wherein, R¹, R², R³, R⁴ and R⁵ are the same as defined above.],
which comprises the below-mentioned step (A) and the step (B) according to [1] or [2],
a step (A): a step of reacting a compound represented by formula (3):

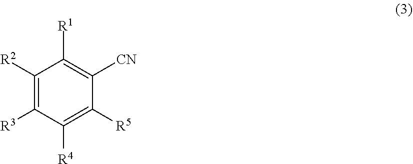

[wherein, R¹, R², R³, R⁴ and R⁵ are the same as defined above.] (hereinafter, referred to as Compound (3)) with a compound represented by formula (4):

CH₃MgX (4)

[wherein, X represents a chlorine atom, a bromine atom, or an iodine atom.] (hereinafter, referred to as Compound (4)) to produce a compound represented by formula (1).
[4] A process for preparing a compound represented by formula (5):

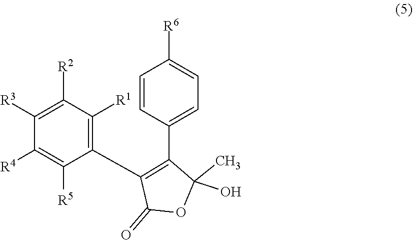

[wherein, R¹, R², R³, R⁴ and R⁵ are the same as defined above, and R⁶ represents a hydrogen atom, a fluorine atom, a chlorine atom, or a bromine atom.] (hereinafter, referred to as Compound (5)),
which comprises the step according to [1] or [2] and the below-mentioned step (C),
a step (C): a step of reacting a compound represented by formula (2):

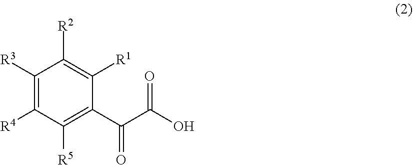

[wherein, R¹, R², R³, R⁴ and R⁵ are the same as defined above.]
with a compound represented by formula (6)

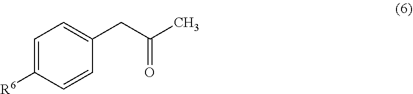

[wherein, R⁶ is the same as defined above.] (hereinafter, referred to as Compound (6))
in the presence of a Lewis acid to produce the compound represented by formula (5).
[5] The process according to [4] wherein the step (C) is conducted in the presence of an alkaline earth metal salt.
[6] A process for preparing a compound represented by formula (7):

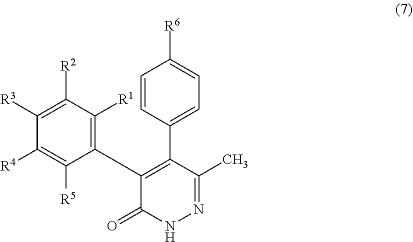

[wherein, R¹, R², R³, R⁴, R⁵ and R⁶ are the same as defined in [4].] (hereinafter, referred to as Compound (7)),
which comprises the step (B) and the step (C) according to [4] or [5] as well as the below-mentioned step (D),

a step (D): a step of reacting a compound represented by formula (5):

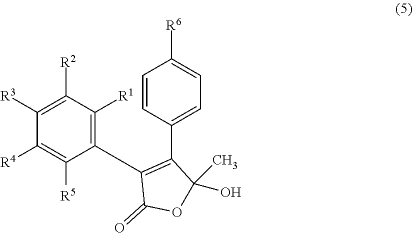

[wherein, R¹, R², R³, R⁴, R⁵ and R⁶ are the same as defined above.]
with hydrazine to produce a compound represented by formula (7).
[7] The process according to [6] wherein the step (D) is conducted in the presence of an alkaline earth metal salt.
[8] A process for preparing a compound represented by formula (8):

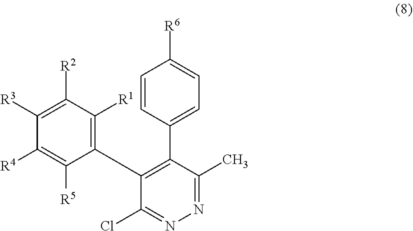

[wherein, R¹, R², R³, R⁴, R⁵ and R⁶ are the same as defined [6].] (hereinafter, referred to as Compound (8)),
which comprises the step (B), the step (C) and the step (D) according to [6] or [7] as well as the below-mentioned step (E),
a step (E): a step of reacting a compound represented by formula (7):

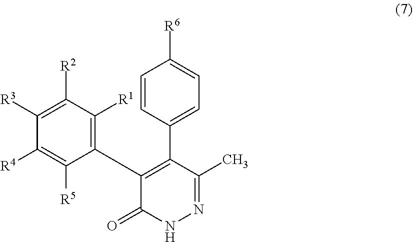

[wherein, R¹, R², R³, R⁴, R⁵ and R⁶ are the same as defined above.]
with a chlorinating agent to produce the compound represented by formula (8).
[9] The process according to [8] wherein the step (E) is conducted in the presence of an alkaline earth metal salt.
[10] The process according to any one of [1] to [5] wherein R¹ and R⁵ each independently represents a fluorine atom, and R², R³ and R⁴ each independently represents a hydrogen atom.
[11] The process according to any one of [6] to [10] wherein R¹ and R⁵ each independently represents a fluorine atom, and R², R³ and R⁴ each independently represents a hydrogen atom, and R⁶ represents a hydrogen atom, a fluorine atom, a chlorine atom, or a bromine atom.

### Effect of Invention

The present invention can produce the compound (2) in good yield. Also the present invention can produce the compound (8) effectively by using the compound (2).

### MODE FOR CARRYING OUT THE INVENTION

Hereinafter, the present invention is explained in more detail.

The compound (1) is explained below.

Examples of the hydrocarbon group which is expressed by R¹, R², R³, R⁴ or R⁵ include an alkyl group having 1 to 20 carbon atom(s) such as methyl group, ethyl group, propyl group, isopropyl group, butyl group, isobutyl group, tert-butyl group, pentyl group, hexyl group, and cycloalkyl group having 3 to 20 carbon atoms such as cyclopentyl group, cyclohexyl group, and norbornyl group. Among them, the alkyl group having 1 to 6 carbon atoms and the cycloalkyl group having 3 to 6 carbon atoms are preferably included, the alkyl group having 1 to 6 carbon atoms, cyclopently group and cyclohexyl group are more preferably included, the alkyl group having 1 to 4 carbon atoms is further preferably included, and methyl group, ethyl group and propyl group are particularly preferably included.

Examples of the hydrocarbon group substituted with halogen atom(s) which is expressed by R¹, R², R³, R⁴ or R⁵ include preferably the above-mentioned alkyl group in which any hydrogen atom(s) in the above-mentioned hydrocarbon group is/are replaced with one or a plural of halogen atom(s), and specific examples thereof include preferably trifluoromethyl group, pentafluoroethyl group, heptafluoropropyl group, difluoromethyl group, fluoromethyl group, dichloromethyl group, chloromethyl group, bromomethyl group, and iodomethyl group, more preferably trifluoromethyl group, pentafluoroethyl group, difluoromethyl group, fluoromethyl group, chloromethyl group, bromomethyl group, and iodomethyl group, further preferably trifluoromethyl group, difluoromethyl group, fluoromethyl group, chloromethyl group, and bromomethyl group, and particularly preferably trifluoromethyl group.

According to one embodiment, at least one of R¹, R², R³, R⁴ and R⁵ includes preferably a halogen atom (such as fluorine atom, chlorine atom, or bromine atom), or the hydrocarbon group substituted with halogen atom(s).

As to R¹ and R⁵, the halogen atom or the hydrocarbon group substituted with halogen atom(s) is more preferably included. Further preferably, a fluorine atom is included and then R², R³ and R⁴ may represent a hydrogen atom.

As to R² and R³, when any one thereof represents a fluorine atom, a chlorine atom, a bromine atom, a hydrocarbon group, or a hydrocarbon group substituted with halogen atom(s), another one thereof represents preferably a hydrogen atom, and then R¹, R⁴ and R⁵ may represent a hydrogen atom.

Next, the step (B) is explained below.

In the step (B), the compound (1) and nitrosyl sulfuric acid are reacted in the presence of water to produce the compound (2).

In the step, the nitrosyl sulfuric acid is usually used within the range of 1 to 10 molar ratio(s), preferably within the range of 2 to 6 molar ratio(s), and more preferably 3 to 5 molar ratio(s), as opposed to 1 mole of the compound (1).

Nitrosyl sulfuric acid is usually used as a sulfuric acid solution (hereinafter, referred to as “sulfuric acid solution of nitrosyl sulfuric acid”) in the reaction. The concentration of the nitrosyl sulfuric acid in the sulfuric acid solution of nitrosyl sulfuric acid is usually within the range of 10 to 60% by weight.

The sulfuric acid solution of nitrosyl sulfuric acid is usually used in the form of a solution containing 3 to 30% by weight as a moisture content. The sulfuric solution of nitrosyl sulfuric acid used contains preferably 4 to 20% by weight, more preferably 5 to 19% by weight, further preferably 10 to 17% by weight, and particularly preferably 14 to 16% by weight.

The nitrosyl sulfuric acid is typically prepared by a method of reacting fuming nitric acid with sulfur dioxide, or a method of reacting chlorosulfuric acid with nitrogen dioxide. Examples of the sulfuric acid solution of nitrosyl sulfuric acid include a commercially available sulfuric acid solution containing 7 to 20% by weight as moisture concentration or a 87% sulfuric acid solution containing 40% nitrosyl sulfuric acid, which may be used as itself, or may be used by adding water, sulfuric acid, or water and sulfuric acid in combination these solutions in advance before applying to the reaction to adjust the applying solution to the above-mentioned preferred moisture concentration. The moisture concentration may be measured by Karl Fisher method.

Examples of the sulfuric acid solution of nitrosyl sulfuric acid to be applied to the reaction include the solutions in which the moisture amount in the sulfuric acid solution of nitrosyl sulfuric acid that is adjusted to the above-mentioned preferred moisture concentration is within the range of usually 1 to 50 molar ratio(s), preferably 6 to 50 molar ratio(s), more preferably 6 to 30 molar ratio(s), further preferably 8 to 13.5 molar ratio(s), and particularly preferably 11 to 13.5 molar ratio(s), as opposed to 1 mole of the compound (1).

The reaction is usually conducted as a certain embodiment wherein the compound (1) is added to the above-mentioned sulfuric solution of nitrosyl sulfuric acid (hereinafter, referred to as Embodiment 1). The reaction is preferably conducted as a certain embodiment wherein when the compound (1) is added to the sulfuric acid solution of nitrosyl sulfuric acid, water is added separately from the sulfuric acid solution of nitrosyl sulfuric acid (hereinafter, referred to as Embodiment 2).

The reaction temperature is within the range of usually 0 to 70° C., preferably 10 to 60° C., and more preferably 20 to 60° C.

When the compound (1) is added to the sulfuric acid solution of nitrosyl sulfuric acid, or when the compound (1) and water are added to the sulfuric acid solution of nitrosyl sulfuric acid, the addition procedure may be conducted all at once or may be conducted each in some divided forms, however, the addition procedure is preferably conducted while an addition rate is adjusted such that the above-mentioned reaction temperature ranges is maintained.

When the compound (1) and water are added separately to the sulfuric acid solution of nitrosyl sulfuric acid, the amount of water to be added separately is within the range of usually 2 to 30 molar ratio(s), preferably 2 to 20 molar ratio(s), and more preferably 2 to 15 molar ratio(s), as opposed to 1 mole of the compound (1).

The reaction period of the reaction may be varied depending on the reaction condition such as the reaction temperature and the like, and is within the range of usually 0.1 to 100 hours, and preferably 1 to 48 hour(s).

Some inert solvent for the reaction may be added to the reaction.

The reaction may be conducted in the presence of silicon dioxide-containing inorganic material. Examples of the silicon dioxide-containing inorganic materials include silica gel, Celite (Registered Trademark), Radiolite (Registered Trademark), diatomaceous earth, and sea sand, and preferably silica gel.

When the reaction is conducted in the presence of the silicon dioxide-containing inorganic material, the amount used thereof is within the range of usually 0.0001 to 10% by weight, and preferably 0.001 to 5% by weight, as opposed to 1 part by weight of the compound (1). The silicon dioxide-containing inorganic material to be added is usually used as a powder form, and its particle size is not specifically limited.

The compound (2) can be isolated and purified according to a conventional method. For example, when the solids are precipitated out, the solids formed after the completion of the reaction can be collected by filtration to isolate the compound (2). Also, for example, the reaction mixture is mixed with water after the completion of the reaction, the mixture is extracted with solvent(s), and the resulting organic layers are then washed, dried, and concentrated under reduced pressure to be able to isolate the compound (2). The solvents to be used for the extraction procedure may be any solvents in which the compound (2) is soluble, but not specifically limited thereto, toluene, xylene, ethylbenzene, 1-methyl-2-pyrrolidone, chlorobenzene, and dichlorobenzene. Also, the compound (2) can be further purified by column chromatography or recrystallization.

The step (A) is explained below.

In the step (A), the compound (3) and the compound (4) are reacted to produce the compound (1).

The reaction is usually conducted in a solvent. The solvents are preferably any solvents that is hardly reacted with the compound (4), and examples of the solvents include ether solvents such as diethyl ether, tetrahydrofuran, methyl tert-butyl ether, cyclopentyl methyl ether, and 1,2-dimethoxyethane; hydrocarbon solvents such as pentane, hexane, heptane, octane, benzene, toluene, xylene, mesitylene, cyclohexane, and cyclopentane; and mixed solvents of two or more solvents.

The amount used of the solvent is usually within the range of 1 to 20 part(s) by weight as opposed to 1 part by weight of the compound (3).

Specific examples of the compound (4) include methyl magnesium chloride, methyl magnesium bromide, and methyl magnesium iodide, preferably methyl magnesium chloride and methyl magnesium bromide, and more preferably methyl magnesium chloride.

The reaction is conducted by mixing the compound (3) with the compound (4). Specific procedures include the followings: the compound (3) is added dropwise to the compound (4); the compound (4) is added dropwise to the compound (3); the compound (3) and the compound (4) are added dropwise to solvent at the same time. Preferred procedure includes those in which the compound (3) is added dropwise to the compound (4).

The period for the dropwise addition is within the range of usually 1 minute to 72 hours, preferably 30 minutes to 48 hours, ad more preferably 1 hour to 24 hours. The reaction temperature at the dropwise addition is within the range of usually 10 to 100° C., preferably 15 to 80° C., and more preferably 20 to 70° C.

After the completion of the dropwise addition, preferably the temperature is kept while stirring the reaction mixture. The temperature to be kept may be maintained or changed the reaction temperature at the dropwise addition, and is within the range of usually 20° C. to 70° C., and preferably 30° C. to 60° C. The period of keeping the temperature is within the range of usually 1 minute to 72 hours, preferably 30 minutes to 48 hours, and more preferably 1 hour to 24 hours.

The amount used of the compound (4) is within the range of usually 1 to 5 molar ratio(s), preferably 1 to 3 molar ratio(s), and more preferably 1 to 2 molar ratio(s), as opposed to 1 mole of the compound (3).

The reaction may be conducted in the presence of metallic salt(s), and examples of the metallic salts include copper(I) chloride and zinc(II) chloride.

When the reaction is completed, the reaction mixture is preferably mixed with water, acids or mixtures thereof to decompose the compound (4) which is remained after the reaction. Specifically, the reaction mixture is preferably mixed with water; acids such as hydrochloric acid, sulfuric acid, nitric acid, phosphoric acid, oxalic acid, and acetic acid; or mixtures of two or more of these. Among them, water, hydrochloric acid, sulfuric acid, phosphoric acid, or mixtures of two or more of these is preferably included, and water, hydrochloric acid, sulfuric acid, or mixtures of two or more of these is more is preferably included. After the mixing, the compound (1) can be isolated and purified according to a conventional method. For example, when the solids are precipitated out, the formed solids are collected by filtration to isolate the compound (1). Also, for example, after the mixture is extracted with solvent(s), the resulting organic layers are washed, dried, and concentrated under reduced pressure to isolate the compound (1). The solvents to be used for the extraction procedure may be any solvents in which the compound (1) is soluble, but not specifically limited thereto, ethers such as diethyl ether, tetrahydrofuran, methyl tert-butyl ether, cyclopentyl methyl ether, and 1,2-dimethoxyethane; hydrocarbon solvents such as pentane, hexane, heptane, octane, benzene, toluene, xylene, mesitylene, cyclohexane, and cyclopentane; halogenated hydrocarbon solvents such as dichloromethane, chloroform, and carbon tetrachloride; aromatic halogenated hydrocarbon solvents such as chlorobenzene, and dichlorobenzene; and mixed solvents of two or more of these solvents. Also, the compound (1) may be further purified by column chromatography or recrystallization.

The step (C) is explained.

In the step (C), the compound (2) and the compound (6) are reacted in the presence of Lewis acid to produce the compound (5).

The reaction is usually conducted in a solvent. Examples of the solvents include polar aprotic solvents, hydrophobic solvents, and mixture of the polar aprotic solvents and the hydrophobic solvents, and the mixture of the polar aprotic solvents and the hydrophobic solvents is preferably included. Examples of the polar aprotic solvents include 1-methyl-2-pyrrolidone, N,N-dimethylformamide, N,N-dimethylacetamide, 1,3-dimethyl-2-imidazolidinone, dimethyl sulfoxide; and mixed solvents of two or more of these solvents. Preferred examples of the solvents include 1-methyl-2-pyrrolidone, N,N-dimethylformamide, N,N-dimethylacetamide, 1,3-dimethyl-2-imidazolidinone, and dimethylsulfoxide, and mixture of two or more of these solvents, and more preferred examples of the solvents include 1-methyl-2-pyrrolidone, and N,N-dimethyl formamide and mixture of two or more of these solvents. The amount used of the polar aprotic solvents is within the range of usually 0.01 to 10 molar ratio(s), preferably 0.1 to 8 molar ratio(s), more preferably 0.5 to molar ratio(s), and further preferably 1 to 3 molar ratio(s), as opposed to 1 mole of the compound (2). Examples of the hydrophobic solvents include aromatic hydrocarbons solvents such as toluene and xylene; aromatic halogenated hydrocarbon solvents such as chlorobenzene and dichlorobenzene; halogenated hydrocarbon solvents such as 1,2-dichloroethane and chloroform; ethers such as tetrahydrofuran, 1,2-dimethoxyethane, diisopropyl ether, and mixture of two or more of these solvents. Preferred examples of the solvents include toluene, xylene, ethylbenzene, chlorobenzene, dichlorobenzene, tetrahydrofuran, and mixture of two or more of these solvents, and more preferred examples of the solvents include toluene, xylene, ethylbenzene, and mixture of two or more of these solvents. The amount used of the hydrophobic solvents is within the range of usually 0.5 to 10 parts by weight, preferably 0.5 to 8 parts by weight, more preferably 0.5 to 5 parts by weight, and further preferably 1 to 3 part(s) by weight, as opposed to 1 part by weight of the compound (2).

Examples of Lewis acid include titanium compounds such as titanium tetrachloride, tetraethyl titanate, and tetraisopropyl titanate; aluminum compounds such as aluminum chloride, aluminum ethoxide, and aluminum isopropoxide; boron compounds such as boron trifluoride, boron trichloride, boron tribromide, boron trifluoride diethyl ether complex, trimethoxy borane, and tris(pentafluorophenyl)borane; zirconium compounds such as zirconium chloride, zirconium tetrapropoxide, and zirconium tetrabutoxide. Among them, titanium compounds are preferably included, and titanium tetrachloride is more preferably included. The Lewis acid may be used as only one kind of these compounds or as mixture of two or more kinds of these compounds.

The amount used of the Lewis acid is within the range of usually 0.01 to 1 molar ratio(s), preferably 0.1 to 1 molar ratio(s), and more preferably 0.1 to 0.3 molar ratios, as opposed to 1 mole of the compound (2).

The reaction is conducted by mixing the compound (2) and the compound (6) in the presence of the Lewis acid. As to the mixing, examples of the order of the mixing include the following methods, but not specifically limited thereto: the compound (6) is added to mixture of the compound (2) and the Lewis acid; the Lewis acid is added to mixture of the compound (2) and the compound (6); the compound (2) is added to mixture of the compound (6) and the Lewis acid. Also, the addition of these compounds may be conducted all at once, may be conducted each in some divided forms, or may be conducted in a dropwise manner. When the addition procedure is conducted in a dropwise manner, the period of the addition is usually within the range of 1 minute to 48 hours.

The reaction temperature is within the range of usually 20 to 150° C., preferably 30 to 130° C., and more preferably 30 to 100° C. The reaction period of the reaction may be varied depending on the reaction condition such as the reaction temperature and the like, and is within the range of usually 0.1 to 200 hours, preferably 1 to 100 hour(s), and more preferably 2 to 72 hours.

The reaction is preferably conducted while removing water that is generated by the reaction. The removal of water can be conducted by the following methods: a dehydrating agent such as molecular sieve and the like is used: solvents are azeotroped with Dean stark apparatus and the like; and the reaction is conducted under reduced pressure.

The reaction may be conducted in the presence of alkaline earth metal salts. Examples of the alkaline earth metal salts include magnesium salt, calcium salt, and barium salt. Examples of anion contained in the salts include fluoride ion, chloride ion, bromide ion, iodide ion, sulfate ion, carbonate ion, acetate ion, oxalate ion, phosphate ion, and oxide ion. Specific examples of the alkaline earth metal salts include magnesium fluoride, calcium fluoride, barium fluoride, magnesium chloride, calcium chloride, barium chloride, magnesium sulfate, calcium sulfate, barium sulfate, magnesium carbonate, calcium carbonate, barium carbonate, magnesium phosphate, calcium phosphate, barium phosphate, magnesium oxide, calcium oxide, and barium oxide, preferably calcium chloride, barium chloride, magnesium sulfate, calcium sulfate, barium sulfate, calcium phosphate, magnesium oxide, and calcium oxide, and more preferably calcium chloride, barium chloride, calcium sulfate, and barium sulfate. Among them, as the examples of the alkaline metal earth salt to be used in the step (C), calcium chloride is preferably included. The alkaline earth metal salts may be anhydrates or hydrates thereof. The use form of the alkaline earth metal salts may be in the form of crystal, powder, granule and bulk, which is not specifically limited thereto.

When the reaction is completed, for example, after the reaction solution is mixed with water, acids or mixture of these, the mixture is extracted with solvents, and the resulting organic layers may be then washed, dried, and concentrated under reduced pressure to isolate the compound (5). Here examples of the solvents to be used in the extraction procedure include aromatic hydrocarbon solvents, aromatic halogenated hydrocarbon solvents, halogenated hydrocarbon solvents, ether solvents, and mixture of two or more of these solvents. Also, the compound (5) can be further purified by column chromatography or recrystallization.

The step (D) is explained.

In the step (D), the compound (5) and hydrazine are reacted to produce the compound (7).

The reaction is usually conducted in a solvent. Examples of the solvents include alcohol solvents such as methanol, ethanol, 1-propanol, and 2-propanol; aromatic hydrocarbon solvents such as toluene, ethylbenzene, and xylene; aromatic halogenated hydrocarbon solvents such as chlorobenzene, and 1,2-dichlorobenzene; ether solvents such as tetrahydrofuran, 1,2-dimethoxyethane, and diisopropyl ether; polar aprotic solvents such as 1-methyl-2-pyrrolidone, N,N-dimethylformamide, N,N-dimethylacetamide, 1,3-dimethyl-2-imidazolidinone, and dimethyl sulfoxide; and mixed solvents of two or more of these solvents; preferred examples of the solvents include toluene, xylene, ethylbenzene, chlorobenzene, 1,2-dichlorobenzene, tetrahydrofuran, 1-methyl-2-pyrrolidone, N,N-dimethylformamide, N,N-dimethylacetamide, 1,3-dimethyl-2-imidazolidinone, dimethyl sulfoxide, and mixture of two or more of these solvents, and more preferred examples of the solvents include toluene, xylene, ethylbenzene, 1-methyl-2-pyrrolidone, N,N-dimethylformamide, and mixture of two or more of these solvents.

The amount used of the solvents is within the range of usually 0.5 to 10 parts by weight, preferably 0.5 to 8 parts by weight, more preferably 0.5 to 5 parts by weight, and further preferably 1 to 3 part(s) by weight, as opposed to 1 part by weight of the compound (5).

Hydrazine may be used in a form of anhydrates or hydrates thereof, however, is usually used in a form of hydrates.

The amount used of hydrazine is within the range of usually 1 to 5 molar ratio(s), and preferably 1 to 3 molar ratio(s), as opposed to 1 mole of the compound (5).

The reaction temperature is within the range of usually 0 to 150° C., preferably 50 to 130° C., and more preferably 60 to 120° C. The reaction period of the reaction may be varied depending on the reaction condition such as reaction temperature and the like, and is within the range of usually 1 to 200 hour(s), preferably 1 to 100 hour(s), more preferably 2 to 72 hours, and further preferably 2 to 24 hours.

The reaction may be conducted while removing water that is generated by the reaction. The removal of water can be conducted by the following methods: a dehydrating agent such as molecular sieve is used: solvents are azeotroped with Dean stark apparatus and the like; and the reaction is conducted under reduced pressure.

The reaction is conducted by mixing the compound (5) with hydrazine. As to the mixing, examples of the order of the mixing include the following methods, but not specifically limited thereto: hydrazine is added to the compound (5); and the compound (5) is added to hydrazine. Also, the addition procedure may be conducted all at once, may be conducted each in some divided forms, or may be conducted in a dropwise manner.

The reaction may be conducted in the presence of alkaline earth metal salts. Examples of the alkaline earth metal salts may be the same as those described in the step (C), and among them, as the examples of the alkaline earth metal salts used in the step (D), barium chloride is preferably included. The alkaline earth metal salts may be anhydrates or hydrates thereof. The use form of the alkaline earth metal salts may be in the form of crystal, powder, granule and bulk, which is not specifically limited thereto.

When the reaction is conducted in the presence of the alkaline earth metal salts, the amount used of the alkaline earth metal salts is within the range of usually 0.0001 to 0.5 molar ratios, preferably 0.001 to 0.3 molar ratios, more preferably 0.01 to 0.2 molar ratios, as opposed to 1 mole of the compound (5).

When the reaction is completed, for example, as needed, the reaction mixture is cooled, and the precipitated out solids are collected by filtration, and the obtained solids are then washed; as needed, after the reaction mixture is cooled, the reaction mixture is then mixed with water, acids, or mixture of these, the precipitated out solids are collected by filtration, and the obtained solids are then washed, and as a result, the compound (7) can be isolated. Examples of the solvents to be used for washing include water; alcohol solvents such as methanol, ethanol, 1-propanol, and 2-propanol; aromatic hydrocarbon solvents such as toluene, ethylbenzene, and xylene; aromatic halogenated hydrocarbon solvents such as chlorobenzene, and 1,2-dichlorobenzene; ether solvents such as tetrahydrofuran, 1,2-dimethoxyethane, and diisopropyl ether; polar aprotic solvents such as 1-methyl-2-pyrrolidone, N,N-dimethyl formamide, N,N-dimethyl acetamide, 1,3-dimethyl-2-imidazolidinone, and dimethyl sulfoxide; and mixture of two or more of these solvents. Also, for example, after the reaction mixture is washed with water, acids, or mixture of these, the mixture is extracted with solvents, and the resulting organic layers are then washed, dried and concentrated under reduced pressure to be able to isolate the compound (7). Examples of the solvents to be used for the extraction procedure include aromatic hydrocarbon solvents, aromatic halogenated hydrocarbon solvents, halogenated hydrocarbon solvents, ether solvents, polar aprotic solvents, and mixture of two or more of these solvents. The compound (7) can be further isolated by column chromatography or recrystallization.

The step (E) is explained.

In the step (E), the compound (7) and a chlorinating agent are reacted to produce the compound (8).

The reaction may be conducted in a solvent or in the absence of solvent. Examples of the solvents include hydrocarbon solvents such as hexane, heptane and octane; aromatic hydrocarbon solvents such as benzene, toluene, xylene, and ethylbenzene: aromatic halogenated hydrocarbon solvents such as chlorobenzene and dichlorobenzene; halogenated hydrocarbon solvents such as 1,2-dichloroethane, and chloroform; ether solvents such as tetrahydrofuran, 1,2-dimethoxyethane, diisopropylether; polar aprotic solvents such as 1-methyl-2-pyrrolidone, N,N-dimethylformamide, N,N-dimethylacetamide, 1,3-dimethyl-2-imidazolidinone, and dimethylsulfoxide; and mixture of two or more of these solvents. Preferred examples of the solvents include toluene, xylene, ethylbenzene, chlorobenzene, dichlorobenzene, tetrahydrofuran, 1-methyl-2-pyrrolidone and mixture of two or more of these solvents, and more preferred examples of the solvents include toluene, xylene, ethylbenzene, 1-methyl-2-pyrrolidone, and mixture of two or more of these solvents. The amount used of the solvents is within the range of usually 0.5 to 10 parts by weight, preferably 0.5 to 8 parts by weight, more preferably 0.5 to 5 parts by weight, and further preferably 1 to 3 part(s) by weight, as opposed to 1 part by weight of the compound (7). The solvents may be used in some divided forms.

Examples of the chlorinating agent include phosphorus oxychloride, phosphorus trichloride, phosphorus pentachloride, phosgene, and mixture of two or more of these compounds, and preferably phosphorus oxychloride.

The amount used of the chlorinating agent is within the range of usually 1 to 10 molar ratio(s), preferably 1 to 5 molar ratio(s), and more preferably 1 to 3 molar ratio(s), as opposed to 1 mole of the compound (7).

The reaction is conducted by mixing the compound (7) with the chlorinating agent. As to the mixing, examples of the order of the mixing include the following methods, but not specifically limited thereto: the chlorinating agent is added to the compound (7); and the compound (7) is added to the chlorinating agent. Also the addition procedure may be conducted all at once, may be conducted each in some divided manner, or may be conducted in a dropwise manner.

The reaction temperature is within the range of usually 0 to 150° C., preferably 50 to 130° C., more preferably to 120° C., and further preferably 80 to 120° C. The reaction period of the reaction may be varied depending on the reaction condition such as the reaction temperature and the like, and is within the range of usually 1 to 200 hour(s), preferably 1 to 100 hour(s), more preferably 2 to 72 hours, and further preferably 2 to 24 hours.

The reaction may be conducted under reduced pressure or atmosphere pressure.

The reaction may be conducted in the presence of alkaline earth metal salts. Examples of the alkaline earth metal salts may be the same as those described in the step (C), and among them, as the examples of the alkaline earth metal salts to be used in the step (E), calcium chloride is preferably included. The alkaline earth metal salts may be anhydrates or hydrates thereof. The use form of the alkaline earth metal salts may be in the form of crystal, powder, granule, and bulk, which is not specifically limited thereto.

When the reaction is conducted in the presence of the alkaline earth metal salts, the amount used of the alkaline earth metal salts is within the range of usually 0.0001 to 0.5 molar ratios, preferably 0.001 to 0.3 molar ratios, and more preferably 0.01 to 0.2 molar ratios, as opposed to 1 mole of the compound (7).

When the reaction is completed, for example, the reaction mixture is mixed with water or aqueous basic solution such as aqueous sodium hydroxide solution (as needed, filter aids may be further mixed), and thereafter, the insoluble materials are removed by filtration, and the resulting filtrates are separated with a separatory funnel, and the resulting organic layers are washed, dried and concentrated under reduced pressure to be able to isolate the compound (8). Also, for example, the reaction mixture is mixed with water or aqueous basic solution (such as aqueous sodium hydroxide solution), and then separated with a separatory funnel, and the resulting organic layers are washed, dried and concentrated under reduced pressure to be able to isolate the compound (8). Examples of the filtrating aids include diatomaceous earth such as Radiolite (Registered Trademark), Celite (Registered Trademark); and activated clay. The compound (8) can be further purified by column chromatography or recrystallization.

### EXAMPLES

Hereinafter, the present invention is explained in more detail by using Examples and Reference Examples, however, the present invention should not be limited to the below-mentioned Examples and the like.

In the below-mentioned examples and the like, unless otherwise specified, a quantitative method was conducted by using high-performance liquid chromatography. The yields of the desired products were calculated based on a peak area of the desired product. The analysis condition is described below.

### [Analysis Condition Using High-Performance Liquid Chromatography]

Internal standard substance: 2-methoxynaphthalene
Mobile phase: A solution: 0.1% aqueous phosphoric acid solution, B solution: acetonitrile
Column: SUMIPAX (Registered Trademark) ODS X-CLUE, particle size 3 μm, 4.6 mm I.D.×100 mm
UV measurement wavelength: 270 nm
Flow rate: 1.0 mL/min

### Column Oven: 40° C.

In the below-mentioned examples, the measurement method of moisture concentration by Karl Fisher method is described below.

### [Measurement Method of Moisture Content by Karl Fisher Method]

The measurement of moisture content was conducted with Coulometric Karl Fisher moisture titrate (AQ-2200, manufactured by Hiranuma Sangyo Co. Ltd.).

### Example 1 (One Example of Embodiment 2 of Step B)

Under nitrogen atmosphere, at room temperature, to nitrosyl sulfuric acid (35% by weight content, sulfuric acid solution) 139.6 g was added water 2.5 g, and it was thereafter confirmed that the moisture concentration was made 15.0% by weight with Karl Fischer moisture titrate. To the resulting mixture was added silica gel 1.5 g, and the mixture was stirred, and 2′,6′-difluoroacetophenone 15.0 g and water 7.5 g were added dropwise separately at the same time at 43° C. over 8 hours thereto, and the mixture was stirred for additional 1 hour. To the resulting mixture was added dropwise water 41.7 g, and the mixture was filtered at 80° C. To the filtrate was added sodium chloride 7.0 g, and the mixture was then extracted with toluene 132.1 g at 80° C. The resulting organic layers were analyzed with high-performance liquid chromatography to confirm that a toluene solution 146.1 g containing the desired product 2,6-difluorobenzoyl formic acid 16.4 g was obtained (the desired product yield: 92%).

### Example 2 (One Example of Embodiment 2 of Step B)

Under nitrogen atmosphere, at room temperature, to nitrosyl sulfuric acid (35% by weight content, sulfuric acid solution) 139.2 g was added water 0.07 g, and it was thereafter confirmed that the moisture concentration was made 14.0% by weight with Karl Fischer moisture titrate. To the resulting mixture was added silica gel 1.5 g, and the mixture was stirred, and 2′,6′-difluoroacetophenone 15.0 g and water 7.5 g were added dropwise separately at the same time at 40° C. over 8 hours thereto, and the mixture was stirred for additional 1 hour. To the resulting mixture was added dropwise water 41.7 g, and the mixture was filtered at 80° C. To the filtrate was added sodium chloride 7.7 g, and the mixture was then extracted with toluene 129.09 g at 80° C. The resulting organic layers were analyzed with high-performance liquid chromatography to confirm that a toluene solution 135.43 g containing the desired product 2,6-difluorobenzoyl formic acid 16.3 g was obtained (the desired product yield: 92%).

### Example 3 (One Example of Embodiment 2 of Step B)

Under nitrogen atmosphere, at room temperature, to nitrosyl sulfuric acid (35% by weight content, sulfuric acid solution) 139.7 g was added water 3.9 g, and it was thereafter confirmed that the moisture concentration was made 16.0% by weight with Karl Fischer moisture titrate. To the resulting mixture was silica gel 1.5 g and the mixture was stirred, and 2′,6′-difluoroacetophenone 15.0 g and water 7.5 g were added dropwise separately at the same time at 40° C. over 8 hours thereto, and the mixture was stirred for additional 1 hour. To the resulting mixture was added dropwise water 41.7 g, and the mixture was filtered at 80° C. To the filtrate was added sodium chloride 7.0 g, and the mixture was then extracted with toluene 131.6 g at 80° C. The resulting organic layers were analyzed with high-performance liquid chromatography to confirm that a toluene solution 142.9 g containing the desired product 2,6-difluoro benzoyl formic acid 16.3 g was obtained (the desired product yield: 92%).

### Example 4 (One Example of Embodiment 2 of Step B)

Under nitrogen atmosphere, at room temperature, to nitrosyl sulfuric acid (40% by weight content, sulfuric acid solution) 121.5 g were added sulfuric acid 3.68 g and water 15.7 g, and it was thereafter confirmed that the moisture concentration was made 17.0% by weight with Karl Fischer moisture titrate. To the resulting mixture was silica gel 1.5 g and the mixture was stirred, and 2′,6′-difluoroacetophenone 15.0 g and water 7.5 g were added dropwise separately at the same time at 40° C. over 8 hours thereto, and the mixture was stirred for additional 1 hour. To the resulting mixture was added dropwise water 41.7 g, and the mixture was filtered at 80° C. To the filtrate was added sodium chloride 8.2 g, and the mixture was then extracted with toluene 128.6 g at 80° C. The resulting organic layers were analyzed with high-performance liquid chromatography to confirm that a toluene solution 120.6 g containing the desired product 2,6-difluoro benzoyl formic acid 15.7 g was obtained (the desired product yield: 89%).

### Example 5 (One Example of Embodiment 2 of Step A)

Under nitrogen atmosphere, to methyl magnesium chloride (3 mol/kg, THF solution) 38.4 g was added dropwise a solution of 2,6-diflurobenzonitrile 10.5 g that was dissolved in toluene 10.6 g over 2 hours while the dropping rate was adjusted such that the reaction temperature was made to the range between 36 to 40° C., and the mixture was stirred at 38 to 39° C. for 5 hours. The resulting mixture was added dropwise to 20% aqueous sulfuric acid solution 92.0 g while the dropping rate was adjusted such that the reaction temperature was made to the range between 27 to 30° C., and thereto was added toluene 11.1 g, and the mixture was stirred at 28° C. for 2.5 hours. The resulting mixture was separated with a separatory funnel and the aqueous layers were removed. To the remaining organic layers was added 5% aqueous sodium bicarbonate solution 31.6 g, and the mixture was separated with a separatory funnel at 30° C. To the resulting organic layers was added water 30.8 g, and the mixture was separated with a separatory funnel at 30° C. The resulting organic layers were analyzed with high-performance liquid chromatography to confirm that the desired product 2′,6′-difluoroacetophenone 10.9 g was contained (the desired product yield: 93%).

### Example 6 (One Example of Step C)

To a solution 150.9 g containing 2,6-difluorobenzoyl formic acid 48.4 g, toluene 50.9 g and 1-methyl-2-pyrrolidone 51.6 g were added anhydrous calcium chloride 3.1 g and titanium tetrachloride 4.9 g, and the pressure in the reactor was reduced to 28 kPa. The temperature of the resulting mixture was risen to 71° C., and to the mixture was then added dropwise a toluene solution 87.2 g containing phenyl acetone 38.4 g over 2 hours, and the mixture was stirred at 71 to 76° C. while hydrating at reflux with Dean stark apparatus. After 25 hours, the pressure in the reactor was returned to atmospheric pressure, and to the resulting mixture was added 20% hydrochloric acid 15.2 g, and the mixture was then separated with a separatory funnel, and the aqueous layers were removed. To the resulting organic layers was added 20% hydrochloric acid 14.6 g, and the mixture was stirred, and the mixture was separated with a separatory funnel. The resulting organic layer was analyzed with high-performance liquid chromatography to confirm that a solution 219.5 g containing the desired product 3-(2,6-difluorophenyl)-5-hydroxy-5-methyl-4-phenyl-2(5H)-furanone 73.9 g was obtained (the desired product yield: 94%).

### Example 7 (One Example of Step D)

To a toluene solution 200 g containing 3-(2,6-diflurophenyl)-5-hydroxy-5-methyl-4-phenyl-2-(5H)-furanone 68.2 g was added barium chloride dihydrates 5.0 g, and the mixture was heated to 100° C. To the resulting mixture was added dropwise hydrazine monohydrate 18 g over 8 hours, and the mixture was stirred for 8 hours, and then cooled to 30° C., and thereto was added water 34.2 g, and the mixture was filtered. The resulting filtrates were washed with methanol 68.4 g and water 68.3 g successively, and then dried. The obtained solids were analyzed with high-performance liquid chromatography to confirm that the desired product 4-(2,6-diflurophenyl)-6-methyl-5-phenyl-3(2H)-pyridazinone (content 94.7%) 67.3 g was obtained (the desired product yield: 96%).

### Example 8 (One Example of Step E)

Under nitrogen atmosphere, 4-(2,6-diflurophenyl)-6-methyl-5-phenyl-3(2H)-pyridazinone 15.0 g (content 94.3%), anhydrous calcium chloride 0.15 g and xylene 30.0 g were mixed, and the temperature of the mixture was risen to 101° C. To the resulting mixture was added dropwise phosphorus oxychloride 11.7 g over 1 hour. The resulting mixture was stirred at 102° C. for 10 hours, and to the mixture was then added xylene 22.5 g, and the mixture was stirred at 80° C. The resulting mixture was added dropwise to a solution that was formed by mixing and stirring 27% aqueous sodium hydroxide solution 35.3 g and Radiolite (Registered Trademark) #700 1.0 g over 30 minutes while an addition rate was adjusted such that the temperature of the reaction solution was within the range of 80 to 85° C., and thereto was then added 27% aqueous sodium hydroxide solution 7.5 g, and as a result, the pH of the aqueous layer of the mixture was adjusted to pH 8.0. The resulting mixture was in advance pre-coated with Radiolite (Registered Trademark) #700 1.3 g, and then filtered with a pressure filter whose temperature was kept at 80° C., and the resulting filtrates were separated with a separatory funnel at 80°. After the aqueous layers were removed, to the remaining organic layer was added water 7.5 g, and the mixture was separated with a separatory funnel at 80° C. The resulting organic layers were analyzed with high-performance liquid chromatography to confirm that the desired product 3-chloro-4-(2,6-difluorophenyl)-6-methyl-5-phenylpyridazinone 14.8 g was obtained (the desired product yield: 99%).

### Example 9 (One Example of Embodiment 1 of Step B)

Under nitrogen atmosphere, at room temperature, to nitrosyl sulfuric acid (42% by weight content, moisture content 7.9%, sulfuric acid solution) 115.8 g was added a dilute sulfuric acid solution that was formed by mixing sulfuric acid 23.16 g and water 13.6 g, and it was thereafter confirmed that the moisture concentration was made 14.9% by weight with Karl Fischer moisture titrate. To the resulting mixture were nitric acid 0.4 g and silica gel 1.5 g, and the mixture was stirred, and 2′,6′-difluoroacetophenone 15.0 g was added dropwise at 43° C. over 8 hours thereto, and the mixture was stirred for additional 1 hour. To the resulting mixture was added dropwise water 34.7 g, and the mixture was filtered at 80° C. To the filtrate was added sodium chloride 6.4 g, and the mixture was then extracted with toluene 128.1 g at 80° C. The resulting organic layers were analyzed with high-performance liquid chromatography to confirm that a toluene solution 125.8 g containing the desired product 2,6-difluorobenzoyl formic acid 16.2 g was obtained (the desired product yield: 91%).

### Example 10

### One Example of Embodiment 1 of Step B

Under nitrogen atmosphere, at room temperature, to nitrosyl sulfuric acid (42% by weight content, moisture content 7.9%, sulfuric acid solution) 115.8 g was added a concentrated sulfuric acid 23.16 g. To the resulting mixture was added silica gel 1.5 g, and the mixture was stirred, and 2′,6′-difluoroacetophenone 15.0 g and water 17.0 g were added dropwise separately at the same time at 43° C. over 15 hours thereto, and the mixture was stirred for additional 1 hour. To the resulting mixture was added dropwise water 34.7 g, and the mixture was filtered at 80° C. To the filtrate was added sodium chloride 6.0 g, and the mixture was then extracted with toluene 128.1 g at 80° C. The resulting organic layers were analyzed with high-performance liquid chromatography to confirm that a toluene solution 124.4 g containing the desired product 2,6-difluorobenzoyl formic acid 15.3 g was obtained (the desired product yield: 84%).

### Example 11 (One Example of Embodiment 1 of Step B)

Under nitrogen atmosphere, at room temperature, to nitrosyl sulfuric acid (35% by weight content, moisture content 14.7%, sulfuric acid solution) 153.0 g was added 2′-fluoroacetophenone 15.0 g at 50° C. over 8 hours, and the mixture was stirred for additional 1 hour. To the resulting mixture was added dropwise water 45.9 g, followed by addition of sodium chloride 7.7 g, and the mixture was extracted with toluene 120.2 g at 80° C. The resulting organic layers were analyzed with high-performance liquid chromatography to confirm that a toluene solution 124.0 g containing the desired product 2-fluorobenzoyl formic acid 14.7 g was obtained (the desired product yield: 83%).

### Example 12 (One Example of Embodiment 1 of Step B)

Under nitrogen atmosphere, at room temperature, to nitrosyl sulfuric acid (35% by weight content, moisture content 14.7%, sulfuric acid solution) 159.1 g was added 4′-methyl acetophenone 15.0 g at 50° C. over 8 hours, and the mixture was stirred for additional 1 hour. To the resulting mixture was added dropwise water 47.8 g, followed by addition of sodium chloride 8.0 g, and the mixture was then extracted with toluene 120.1 g at 80° C. The resulting organic layers were analyzed with high-performance liquid chromatography to confirm that a toluene solution 126.5 g containing the desired product 4-methylbenzoyl formic acid 13.5 g was obtained (the desired product yield: 75%).

### Example 13 (One Example of Embodiment 1 of Step B)

Under nitrogen atmosphere, at room temperature, to nitrosyl sulfuric acid (35% by weight content, moisture content 14.7%, sulfuric acid solution) 178.6 g was added dropwise acetophenone 15.0 g at 50° C. over 8 hours, and the mixture was stirred for additional 1 hour. To the resulting mixture was added dropwise water 53.6 g, followed by addition of sodium chloride 9.0 g, and the mixture was extracted with toluene 120.2 g at 80° C. The resulting organic layers were analyzed with high-performance liquid chromatography to confirm that a toluene solution 127.4 g containing the desired product benzoyl formic acid 15.2 g was obtained (the desired product yield: 83%).

### Example 14 (One Example of Embodiment 1 of Step B)

Under nitrogen atmosphere, at room temperature, to nitrosyl sulfuric acid (35% by weight content, moisture content 14.7%, sulfuric acid solution) was added dropwise 2′-chloroacetophenone 15.0 g at 50° C. over 8 hours, and the mixture was stirred for additional 1 hour. To the resulting mixture was added dropwise water 41.0 g, followed by addition of sodium chloride 6.9 g, and the mixture was extracted with toluene 120.2 g at 80° C. The resulting organic layers were analyzed with high-performance liquid chromatography to confirm that a toluene solution 122.7 g containing the desired product 2-chlorobenzoyl formic acid 9.9 g was obtained (the desired product yield: 57%).

### Example 15 (One Example of Embodiment 1 of Step B)

At nitrogen atmosphere, at room temperature, to nitrosyl sulfuric acid (35% by weight content, moisture content 14.7%, sulfuric acid solution) was added dropwise 3′-chloroacetophenone 15.0 g at 50° C. over 8 hours, and the mixture was stirred for additional 1 hour. To the resulting mixture was added dropwise water 41.1 g, followed by addition of sodium chloride 6.9 g, and the mixture was then extracted with toluene 120.2 g at 80° C. The resulting organic layers were analyzed with high-performance liquid chromatography to confirm that a toluene solution containing the desired product 3-chlorobenzoyl formic acid 16.0 g was obtained (the desired product yield: 92%).

### Example 16 (One Example of Embodiment 1 of Step B)

At nitrogen atmosphere, at room temperature, nitrosyl sulfuric acid (35% by weight content, moisture content 14.7%, sulfuric acid solution) 136.8 g was added dropwise 4′-chloroacetophenone 15.0 g at 50° C. over 8 hours, and the mixture was stirred for additional 1 hour. To the resulting mixture was added dropwise water 41.1 g, followed by addition of sodium chloride 6.9 g, and the mixture was then extracted with toluene 120.2 g at 80° C. The resulting organic layers were analyzed with high-performance liquid chromatography to confirm that a toluene solution 131.1 g containing the desired product 4-chlorobenzoyl formic acid 15.4 g was obtained (the desired product yield: 88%).

### Example 17 (One Example of Embodiment 1 of Step B)

At nitrogen atmosphere, at room temperature, to nitrosyl sulfuric acid (35% by weight content, moisture content 14.6%, sulfuric acid solution) 112.7 g was added dropwise 4′-trifluoromethyl acetophenone 15.0 g at 50° C. over 8 hours, and the mixture was stirred for additional 1 hour. To the resulting mixture was added dropwise water 33.8 g, followed by addition of sodium chloride 5.6 g, and the mixture was extracted with toluene 120.2 g at 80° C. The resulting organic layers were analyzed with high-performance liquid chromatograph to confirm that a toluene solution 124.1 g containing the desired product 4-trifluoromethylbenzoyl formic acid 14.3 g was obtained (the desired product yield: 87%).

### Example 18 (One Example of Embodiment 1 of Step B)

At nitrogen atmosphere, at room temperature, to nitrosyl sulfuric acid (35% by weight content, moisture content 14.6%, sulfuric acid solution) 107.6 g was added dropwise 3′-bromoacetophenone 15.0 g at 50° C. over 8 hours, and the mixture was stirred for additional 1 hour. To the resulting mixture was added dropwise water 32.3 g, followed by addition of sodium chloride 5.4 g, and the mixture was then extracted with toluene 120.2 g at 80° C. The resulting organic layers were analyzed with high-performance liquid chromatography to confirm that a toluene solution 130.1 g containing the desired product 3-bromobenzoyl formic acid 14.9 g was obtained (the desired product yield: 88%).

### Reference Example (Preparation of Acetophenone)

Phenyl acetic acid 39.2 g was dissolved in acetic anhydride 30.3 g at 40° C. to produce a solution. The resulting solution that was kept at 40° C. and 1-methyl imidazole 11.9 g were added dropwise separately at the same time to acetic anhydride 30.3 g at 25° C., and the mixture was then stirred for 24 hours. To the resulting mixture was added water 5.2 g. The pressure in the reactor was reduced to 5 kPa, and the internal temperature in the reactor was raised to 80° C., and the distilled fractions were removed. The pressure in the reaction was further reduced to 2 kPa, and the internal temperature in the reactor was raised to 130° C. to produce a solution 75.3 g containing phenyl acetone. The solution 75.3 g containing phenyl acetone, toluene 37.0 g, and water 18.5 g were mixed, and thereto was then added dropwise 27% aqueous sodium hydroxide solution 46.9 g such that the pH of the aqueous layers of the mixture was adjusted to pH 6.2. After the aqueous layers were removed, the resulting organic layers were analyzed with gas chromatography to confirm that a toluene solution 70.4 g containing phenyl acetone 30.9 g was obtained (the desired product yield: 80%).

1. A process for preparing a below-mentioned compound represented by formula (2), which comprises a step (B): a step of reacting a compound represented by formula (1): 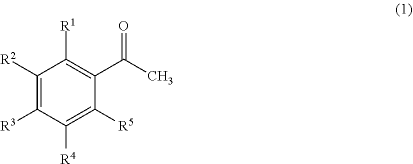
wherein, R¹, R², R³, R⁴ and R⁵ each independently represents a fluorine atom, a chlorine atom, a bromine atom, a hydrogen atom, a hydrocarbon group, or a hydrocarbon group substituted with halogen atom(s),
with a nitrosyl sulfuric acid in the presence of water to produce the compound represented by formula (2): 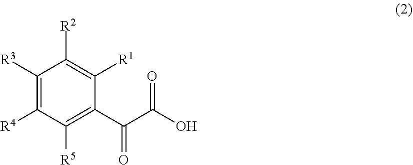
wherein, R¹, R², R³, R⁴ and R⁵ are the same as defined above.

2. The process according to claim 1 wherein the step (B) is conducted in the presence of a silicon dioxide-containing inorganic material.

3. A process for preparing a below-mentioned compound represented by formula (2): 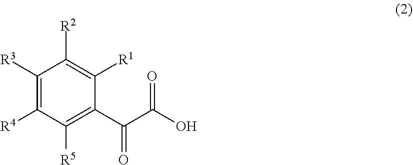
wherein, R¹, R², R³, R⁴ and R⁵ are the same as defined above,
which comprises the below-mentioned step (A) and the step (B) according to claim 1,
a step (A): a step of reacting a compound represented by formula (3): 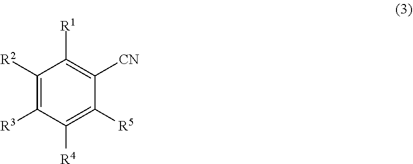
wherein, R¹, R², R³, R⁴ and R⁵ are the same as defined above,
with a compound represented by formula (4):
CH₃MgX (4)
wherein, X represents a chlorine atom, a bromine atom, or an iodine atom,
to produce a compound represented by formula (1).

4. A process for preparing a compound represented by formula (5): 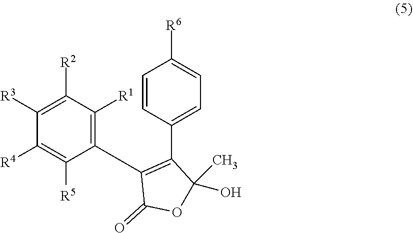
wherein, R¹, R², R³, R⁴ and R⁵ are the same as defined above, and R⁶ represents a hydrogen atom, a fluorine atom, a chlorine atom, or a bromine atom,
which comprises the step according to claim 1 and the below-mentioned step (C),
a step (C): a step of reacting a compound represented by formula (2): 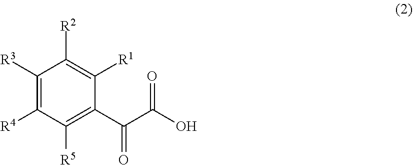
wherein, R¹, R², R³, R⁴ and R⁵ are the same as defined above,
with a compound represented by formula (6) 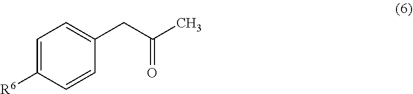
wherein, R⁶ is the same as defined above,
in the presence of a Lewis acid to produce the compound represented by formula (5).

5. The process according to claim 4 wherein the step (C) is conducted in the presence of an alkaline earth metal salt.

6. A process for preparing a compound represented by formula (7): 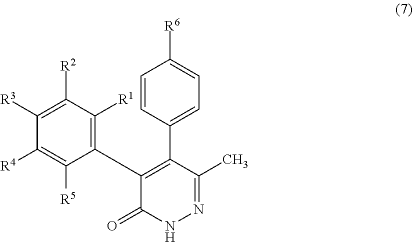
wherein, R¹, R², R³, R⁴, R⁵ and R⁶ are the same as defined in [4],
which comprises the step (B) and the step (C) according to claim 4 as well as the below-mentioned step (D),
a step (D): a step of reacting a compound represented by formula (5): 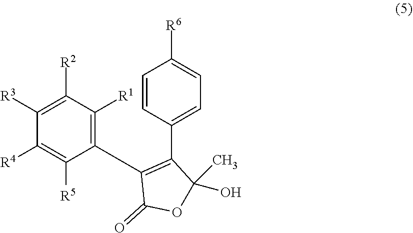
wherein, R¹, R², R³, R⁴, R⁵ and R⁶ are the same as defined above,
with hydrazine to produce a compound represented by formula (7).

7. The process according to claim 6 wherein the step (D) is conducted in the presence of an alkaline earth metal salt.

8. A process for preparing a compound represented by formula (8): 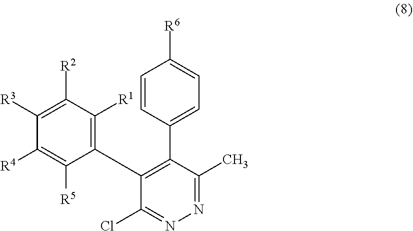
wherein, R¹, R², R³, R⁴, R⁵ and R⁶ are the same as defined [6],
which comprises the step (B), the step (C) and the step (D) according to claim 6 as well as the below-mentioned step (E),
a step (E): a step of reacting a compound represented by formula (7): 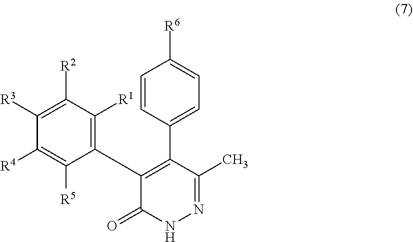
wherein, R¹, R², R³, R⁴, R⁵ and R⁶ are the same as defined above,
with a chlorinating agent to produce the compound represented by formula (8).

9. The process according to claim 8 wherein the step (E) is conducted in the presence of an alkaline earth metal salt.

10. The process according to claim 1 wherein R¹ and R⁵ each independently represents a fluorine atom, and R², R³ and R⁴ each independently represents a hydrogen atom.

11. The process according to claim 6 wherein R¹ and R⁵ each independently represents a fluorine atom, and R², R³ and R⁴ each independently represents a hydrogen atom, and R⁶ represents a hydrogen atom, a fluorine atom, a chlorine atom, or a bromine atom.
